(19) United States
(12) Patent Application Publication
(10) Pub. No.: US 2024/0325608 A1
(43) Pub. Date: Oct. 3, 2024

(54) DELIVERY SYSTEM AND METHOD FOR USING SAME

(71) Applicant: COLLAGEN MATRIX, INC., Oakland, NJ (US)

(72) Inventors: Hui-Chen Chen, Wayne, NJ (US); Natsuyo Shishido Lee, Basking Ridge, NJ (US); Daniel Ammon, Webster, NY (US)

(73) Assignee: Collagin Matrix, Inc., Oakland, NJ (US)

(21) Appl. No.: 18/292,535

(22) PCT Filed: Jul. 29, 2021
(86) PCT No.: PCT/US2021/043747

§ 371 Date: Jan. 26, 2024

(51) Int. Cl. A61L 27/56; A61K 35/28; A61K 38/18; A61K 38/30; A61L 27/22; A61L 27/26; A61L 27/36

(52) U.S. Cl. CPC A61L 27/56; A61K 35/28; A61K 38/1808; A61K 38/1825; A61K 38/1841; A61K 38/185; A61K 38/1858; A61K 38/1866; A61K 38/1875; A61K 38/30; A61L 27/227; A61L 27/26; A61L 27/3604; A61L 2300/414

(57) ABSTRACT

A delivery system for delivering at least one biological substance to a diseased or damaged area of a patient and having a sponge including a top sponge portion with a plurality of protrusions extending downwardly therefrom and a bottom sponge portion having a plurality of wells formed therein, the wells being configured to receive the at least one biological substance therein. Each of the plurality of wells is dimensioned to receive one of the plurality of protrusions therein upon placing the top sponge portion onto the bottom sponge portion to assemble the sponge. Also disclosed is a method for delivering the biological substance(s) to the diseased or damaged area of a patient using the delivery system, and a kit including the biological substance(s) and the delivery system.

### FIELD OF THE INVENTION

The present disclosure relates to systems and methods for delivery of a growth factors, bone marrow aspirate and graft tissue (e.g., autograft) to a diseased or damaged area of a patient.

### BACKGROUND OF THE INVENTION

Growth factors have therapeutic attributes, and are therefore utilized in the treatment of diseased or damaged area of a patient. Such growth factors include, for example, human recombinant growth factors, such as bone morphogenetic proteins (BMPs), PDGF, BDNF, EGF. Such growth factors are found in the human body and help regulate biological processes. The use of BMPs is known in orthopedic applications, such as, for example, introducing BMPs into a diseased or damaged area of a patient's skeleton, e.g., vertebrae of the spine, or other musculoskeletal tissues. Effective ways to deliver growth factors to tissues within the patient's body is therefore desirable.

### SUMMARY OF THE INVENTION

In an exemplary embodiment, the present invention includes a delivery system for delivering at least one biological substance to a diseased or damaged area of a patient. The delivery system comprises a sponge including a top sponge portion having a top base and a plurality of protrusions extending downwardly therefrom; and a bottom sponge portion having a bottom base and a plurality of wells formed therein, the wells being configured to receive the at least one biological substance therein. Each of the plurality of wells is dimensioned to receive one of the plurality of protrusions therein upon placing the top sponge portion onto the bottom sponge portion to assemble the sponge.

In another exemplary embodiment, the present invention includes a method for delivering at least one biological substance to a diseased or damaged area of a patient. The method comprises the steps of providing a sponge, including a top sponge portion having a top base 12a and a plurality of protrusions 14 extending downwardly therefrom, and a bottom sponge portion having a bottom base 12b and a plurality of wells 16 formed therein, the wells being configured to receive the at least one biological substance 22 therein, each of the plurality of wells 16 being dimensioned to receive one of the plurality of protrusions therein upon placing the top sponge portion onto the bottom sponge portion; separating the top sponge portion and bottom sponge portion; adding the at least one biological substance to the plurality of wells 16 of the bottom sponge portion 10b; placing the top sponge portion onto the bottom sponge portion to assemble the sponge, such that the protrusions 14 insertably engage the corresponding wells 16, whereby at least a portion of the at least one biological substance flows into the protrusions 14; and surgically implanting the assembled sponge into the diseased or damaged area of the patient, whereby the at least one biological substance is released from the wells 16 through their pores 20, and through the pores 18 of the protrusions 14, and ultimately out of the sponge 10, into the patient's designated diseased or damaged area and the surrounding tissues, wherein it provides at least one positive therapeutic effect.

In still another exemplary embodiment, the present invention includes a kit for delivering at least one biological substance to a diseased or damaged area of a patient. The kit comprises the at least one biological substance and a delivery system for delivering the at least one biological substance to the diseased or damaged area of a patient. In one embodiment, the delivery system comprises a sponge, including a top sponge portion having a top base 12a and a plurality of protrusions 14 extending downwardly therefrom; and a bottom sponge portion having a bottom base 12b and a plurality of wells 16 formed therein, the wells being configured to receive the at least one biological substance 22 therein. Each of the plurality of wells 16 is dimensioned to receive one of the plurality of protrusions therein upon placing the top sponge portion onto the bottom sponge portion to assemble the sponge.

This Summary of the Invention is neither intended nor should it be construed as being representative of the full extent and scope of the present disclosure. The present disclosure is set forth in various levels of detail in the Summary of the Invention as well as in the attached drawings and the Detailed Description of the Invention, and no limitation as to the scope of the present disclosure is intended by either the inclusion or non-inclusion of elements, components, etc. in this Summary of the Invention. Additional aspects of the present disclosure will become more readily apparent from the Detailed Description, particularly when taken together with the drawings.

The above-described benefits, embodiments, and/or characterizations are not necessarily complete or exhaustive, and in particular, as to the patentable subject matter disclosed herein. Other benefits, embodiments, and/or characterizations of the present disclosure are possible utilizing, alone or in combination, as set forth above and/or described in the accompanying figures and/or in the description herein below. However, the Detailed Description of the Invention, the drawing figures, and the exemplary claim set forth herein, taken in conjunction with this Summary of the Invention, define the disclosure.

### BRIEF DESCRIPTION OF THE DRAWINGS

The accompanying drawings, which are incorporated in and constitute a part of the specification, illustrate embodiments of the disclosure and together with the general description of the disclosure given above and the detailed description of the drawings given below, serve to explain the principles of the disclosures.

It should be understood that the drawings are not necessarily to scale. In certain instances, details that are not necessary for an understanding of the disclosure or that render other details difficult to perceive may have been omitted. It should be understood, of course, that the disclosure is not necessarily limited to the particular embodiments illustrated herein.

FIG. 1A is a top perspective view of a growth factor delivery system according to an embodiment of the present invention.

FIG. 1B is an exploded top perspective view of the growth factor delivery system of FIG. 1A, showing the interior components thereof.

FIG. 1C is an exploded cross-sectional view of the growth factor delivery system shown in FIG. 1B, as taken along lines 1C-1C.

FIG. 1D is a cross-sectional view of the growth factor delivery system of FIG. 1A, as taken along lines 1D-1D.

FIG. 2 is a detailed view of a portion of the growth factor delivery system shown in FIG. 1D.

FIG. 3 is a flowchart showing a method of using the growth factor delivery system according to an embodiment of the present invention.

### DETAILED DESCRIPTION

The present disclosure relates to systems and methods for delivery of growth factors, bone marrow aspirate and/or graft tissue (e.g., autograft) to a diseased or damaged area of a patient.

In various embodiments, the system includes a two-part collagen sponge or collagen-based composite sponge configured to receive, hold and deliver growth factors, bone marrow aspirate and/or graft tissue, as described further herein.

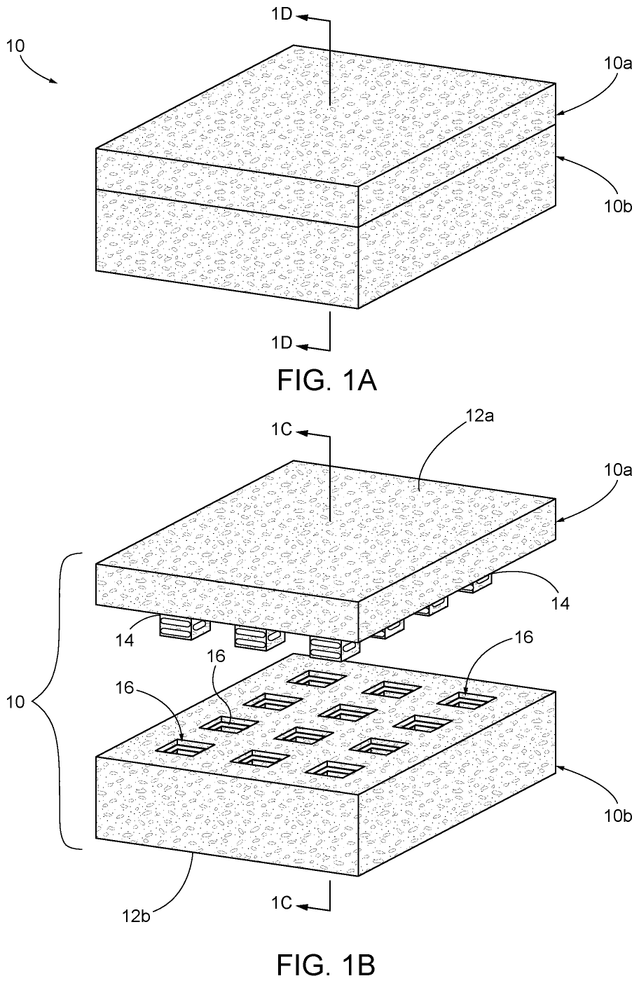

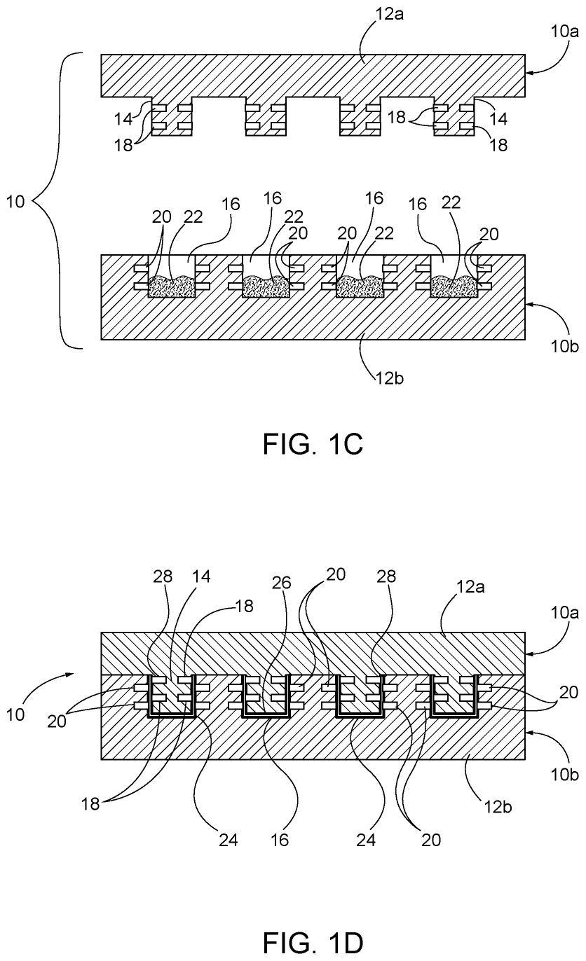

Referring now to FIGS. 1A, 1B, 1C and 1D, an exemplary embodiment of the disclosed delivery system is shown and includes a sponge 10 that has two cooperating/complimentary portions 10a, 10b. The first, or “top” portion 10a includes a top base 12a and a plurality of protrusions 14 extending downwardly therefrom. The second, or “bottom” portion 10b includes a bottom base 12b and a plurality of wells 16 formed therein. The number of protrusions 14 is equal to the number of wells 16. Further, the wells 16 are each dimensioned to receive one of the protrusions 14 therein.

With further reference to FIGS. 1C and 1D, each of the protrusions 14 defines one or more pores 18 within its outer surface (i.e., the portions of the top base 12a that surround and define the protrusions 14). Each of the wells 16 also defines one or more pores 20 in its walls (i.e., the portions of the bottom base 12b that surround and define the wells 16). As discussed further below, the number and size of the pores 18, 20 may vary in different embodiments, depending on the specific medical/therapeutic application

In one example the pores 18, 20 have a density ranging from about 0.05 g/cm³ to about 1.3 g/cm³, a pore size ranging from about 0.1 μm to about 1,500 μm, and a spacing between pores ranging from about 0.1 μm to about 2,000 μm.

In another example, pores 18, 20 have a pore size of 50-500 μm (e.g., 50-200 μm or 150-500 μm), and the collagen or collagen-based composite of the sponge 10 has a density in the range of 0.02-0.8 g/cm³ (e.g., 0.03-0.7 g/cm³).

In another example, pores 18, 20 have a pore size of 25-500 μm (e.g., 50-400 μm or 75-250 μm), and the collagen or collagen-based composite of the sponge 10 has a density range of 0.05 0.8 g/cm³ (e.g., 0.1-0.6 g/cm³).

The wells 16 are configured to receive at least one biological substance 22 therein (e.g., a solution containing the biological substance(s)) when the top and bottom portions 10a, 10b are separated. The biological substance includes growth factors, BMA and/or graft tissue (e.g., autograft, allograft or xenograft). As discussed above, growth factors used with the system of the present disclosure include, for example, human recombinant growth factors, such as bone morphogenetic proteins (BMPs) Other growth factors include, for example; fibroblast growth factors (FGF), insulin like growth factors (IGFs), platelet derived growth factors (PDGF), plasma rich growth factors (PRGF), transforming growth factor-β (TGF-β), epidermal growth factor (EGF), nerve growth factor (NGF) and vascular endothelial growth factors (VEGF). The biological substance 22 is placed into the wells 16, and then the top portion 10a is placed back on top of the bottom portion 10b, as shown in FIG. 1D, and further described below.

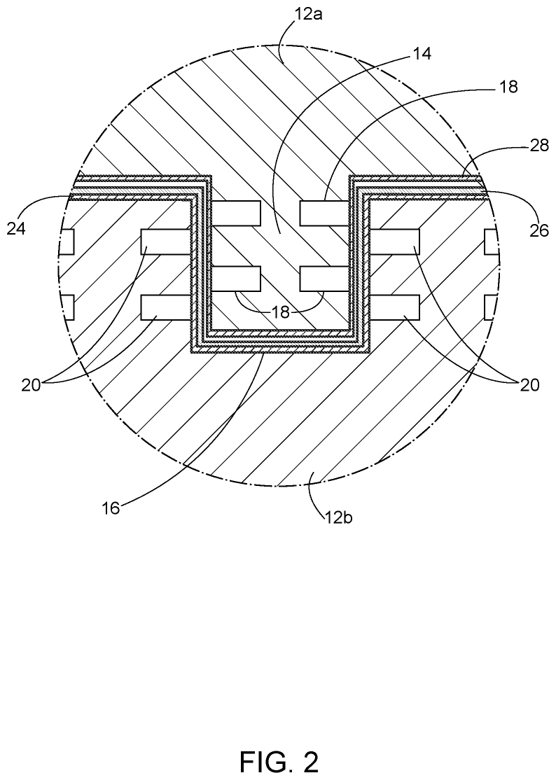

In various embodiments, the wells 16 are lined with one or more collagen barrier layers, or membranes, 24 (see FIG. 1D). More particularly, portions of the bottom base 12b that define and border the wells 16 are provided with one or more collagen barrier layers 24. This collagen barrier layer 24 is shown in detail in FIG. 2. In one embodiment, the wells contain pores, as described above. In another embodiment, the wells do not contain pores but the collagen barrier layers 24 are configured to control the release of biological substance 22.

In various embodiments, the portions of the bottom base 12b that define and border the wells 16 are lined with one or more collagen barrier layers 24, as described above, and one or more overlying absorbent collagen layers, or membranes, 26 that is formed on top of the collagen barrier layer 24, as also illustrated in FIGS. 1D and 2. In one embodiment, the wells contain pores, as described above. In another embodiment, the wells do not contain pores but the collagen barrier layers 24 and/or the overlying absorbent collagen layers 26 are configured to control the release of the biological substance 22.

In various embodiments, portions of the top base 12a that define and border the protrusions 14 are also lined with one or more collagen barrier layers, or membranes, 28, as also illustrated in FIGS. 1D and 2. The collagen barrier layer(s) 28 may be the same as, or similar to, the collagen barrier layer(s) 24 of the bottom base 12b/wells 16.

As disclosed in U.S. Pat. No. 10,279,077, the coating of the membrane can be accomplished by spraying the surface with acid dispersed collagen fibers (e.g., dispersed in lactic acid) at a pH of 2.3-2.5, or alkaline dispersed collagen fibers (e.g., dispersed in NaOH) at a pH of 11 to 12. Any commercial sprayer (e.g., Badger Airbrush Model 150) can be used to spray the dispersed collagen fibers onto the fibrous side of the membrane. Spraying can be performed at 10-60 psi using a nozzle size of 0.1-1.0 mm. The concentration of collagen fibers in the spraying solution ranges from about 0.05% to about 0.1%(w/v) such that the collagen is sprayed in the form of a mist that coats the membrane surface uniformly despite any microscopic irregularities that may exist. A single layer or multiple layers of a collagen coating can be applied via this spraying technique. Typically, the thickness of the collagen coating is in the range of 5-50 μm.

Alternatively, the fibrous surface can be coated with an acid dispersed collagen using a brush. The concentration of collagen used for brush coating can be significantly higher than that used for spraying, ranging from about 0.1% to about 0.9%(w/v). The collagen coating applied using a brush is significantly thicker than that applied by spraying, ranging from about 50 μm to about 100 μm.

Further, the membrane can be dipped into a collagen dispersion in order to coat the fibrous surface of the membrane.

Irrespective of the collagen coating technique employed, the coated membrane is dried so as to more strongly integrate collagen with the collagen-based delivery system surface.

The collagen barrier placed in the wells can be tailored to various hydrophilic/lipophilic balance (HLB) so that it can control the diffusion of the growth factors, BMA or graft tissue (e.g., autograft) placed in the well. The barrier can also be completely hydrophobic as to completely contain the growth factors, BMA or graft tissue (e.g., autograft) in the wells but contain pores within the wells in which the pores now control the diffusion. A combination of pores and coating HLB can also control the diffusion of the growth factors, BMA or graft tissue (e.g., autograft) tissue placed in the well.

The collagen barrier layers in the wells can be hydrophobic thereby containing the biological substance, in solution, in the wells. The release of the biological substance would be solely dependent on the pore size or number of pores in the wells. A more hydrophilic coating that swells enough, to in of itself, to allow for the biological substance(s) to release/diffuse from the wells. The hydrophilic coating can control the release of the biological substance(s) by way of coating thickness or the HLB (hydrophilic/lipophilic balance). A hydrophilic coating in the wells that can release the biological substance(s) can also be engineered with pores as well to further control the release of growth factors. Furthermore, the pores in the wells can be filled with a dissolvable material to delay the release of the biological substance(s).

For the sake of clarity and to focus on other features of the sponge 10, the collagen barrier layers 24, 28 and absorbent collagen layer 26 are not shown in FIG. 1C. However, these collagen layers are shown in FIGS. 1D and 2, as discussed above.

In various embodiments, the collagen barrier layers 24, 28 and/or absorbent collagen layer 26 are formed according to the methods and using the materials disclosed in U.S. Pat. Nos. 8,298,584, 10,071,184, 10,279,077 and/or 10,537,417, all of which are incorporated herein in their entireties. These patents all refer to the collagen layers as “membranes”, as further discussed below.

In exemplary embodiments disclosed in U.S. Pat. No. 8,298,584, a biopolymeric membrane includes at least two layers: (1) a first layer including randomly oriented fibers of a first biopolymer (e.g., collagen), the fibers having a length of 2.5-50 cm (e.g., 10-40 cm); and (2) a second layer including fibers of a second biopolymer (e.g., collagen), the fibers having a length of 0.05-3 mm (e.g., 0.1-1.0 mm). The membrane has a draping angle of 30-90° (e.g., 45-90° or 60-90°, a hydrothermal shrinkage temperature of 48-72° C. (e.g., 50-70° C.), and is permeable to molecules having a molecular weight less than 100,000 Daltons (e.g., <70,000 Daltons or <29,000 Daltons). It can further contain one or more bioactive agents, e.g., various growth factors and antibiotics. The biopolymeric membrane may have a thickness ranging from 0.1 mm to 3.0 mm (e.g., 0.3-1.5 mm, or 0.3-0.7 mm), and a density of 0.04-0.6 g/cm³ (e.g., 0.1-0.4 g/cm³ or 0.2-0.4 g/cm³). U.S. Pat. No. 8,298,584 further discloses a method of making the biopolymeric membrane described above. The method includes the following steps: (i) dispersing fibers of a first biopolymer (e.g., collagen) in an acidic or alkaline solution to form a mixture; (ii) neutralizing, if necessary, the mixture to a pH equal to the isoelectric point of the first biopolymer to produce reconstituted fibers of the first biopolymer; (iii) dehydrating the reconstituted fibers of the first biopolymer; (iv) compressing the dehydrated fibers to form a first layer; (v) freeze-drying the first layer; (vi) coating the freeze-dried first layer with fibers of a second biopolymer (e.g., collagen) to produce a membrane, the fibers of the second biopolymer forming a second layer on the first layer; and (vii) crosslinking the fibers of the first biopolymer and the fibers of the second biopolymer in the membrane. When collagen is used as the first biopolymer, the collagen fibers are dispersed in a solution having a pH value either lower than 3 or higher than 11 to form a mixture. The mixture is then neutralized to a pH of 4.5-6 (e.g., 5.0-5.5) to produce reconstituted collagen fibers.

In exemplary embodiments disclosed in U.S. Pat. Nos. 10,071,184 and 10,279,077, a method is provided for preparing a tissue-based coated membrane resulting in a membrane that is smooth on both sides, so as to minimize tissue adhesion. The method for producing a smooth tissue-based coated membrane includes the following six key steps. First, the fibrous, i.e., parietal, side of an isolated tissue-based membrane is scraped to remove adhering fibers. Second, the tissue-based membrane is processed with an enzyme and chemicals to remove cells, lipids, and extractable blood and non-collagenous molecules. Third, the processed tissue-based membrane is freeze dried. Fourth, the fibrous side of the tissue-based membrane is compressed. Fifth, the compressed fibrous side of the tissue-based membrane is coated with collagen. Finally, the collagen coated tissue-based membrane is exposed to a crosslinking agent.

U.S. Pat. No. 10,537,417 discloses an example featuring a density gradient biopolymeric matrix implant. The implant includes a first homogeneous matrix layer and a second homogeneous matrix layer having a density different from that of the first homogeneous matrix layer. Biopolymeric fibers at the surface of the first homogeneous matrix layer are physically in contact with and cross-linked to the biopolymeric fibers at the surface of the second homogeneous matrix layer. Also disclosed is a three-dimensional density gradient biopolymeric matrix implant that includes a first homogeneous matrix surrounding a second homogeneous matrix having a different density. Biopolymeric fibers at an inner surface of the first homogeneous matrix are physically in contact with and cross-linked to biopolymeric fibers at an outer surface of the second homogeneous matrix.

In various embodiments, the collagen of each of collagen barrier layers 24, 28 and/or absorbent collagen layer 26 is type I collagen, type II collagen, type III collagen or a combination of two or more of these collagen types.

In various embodiments, the sponge 10 of the system is formed from collagen. In various embodiments, the collagen constitutes type I collagen, type II collagen, type III collagen or a combination of two or more of these collagen types.

In various embodiments, the sponge 10 of the system is formed from a collagen-based composite. Such composites include, for example, the following:
1. Collagen/Mineral
2. Collagen/Mineral/BioGlass
3. Collagen/BioGlass

In various embodiments, the collagen/mineral composite sponge has the following distributions (in weight percentage) of collagen fibers and mineral:
5% collagen and 95% mineral;
10% collagen and 90% mineral;
15% collagen and 85% mineral;
20% collagen and 80% mineral;
25% collagen and 75% mineral; and
30% collagen and 70% mineral.

In various embodiments, the collagen/mineral composite sponge has the following ranges of distributions (in weight percentage):
Collagen 40%-90%
BioGlass 10%-90%
Mineral 10%-90%

In various embodiments, the collagen composite sponge also includes one or more glycosaminoglycans (GAGs). The distribution/content of GAGs ranges from 0.05%-10%(in weight percentage) in such embodiments.

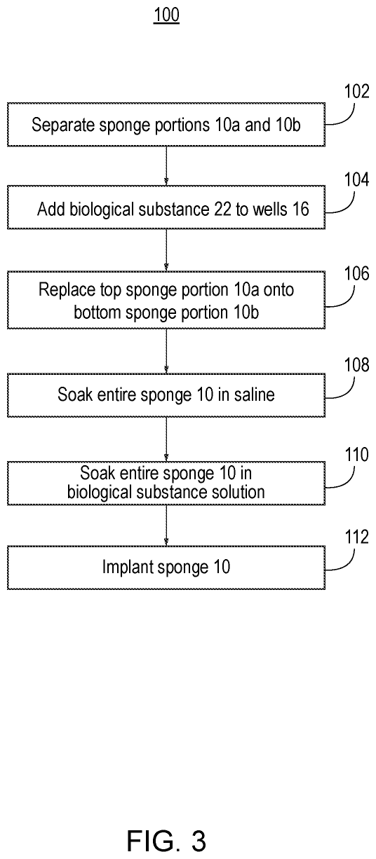

Methods of using the system of the sponge 10 in a patient will now be described, with reference to the flowchart 100 of FIG. 3.

First, the sponge 10 is provided (i.e., prior to a surgical procedure in which the sponge 10 will be implanted into a patient), and the top and bottom sponge portions 10a and 10b are separated 102 from each other.

Next, at least one biological substance 22 (e.g., a solution containing biological substance) is added 104 to the wells 16 of the bottom sponge portion 10b.

The top sponge portion 10a is then replaced 106 on the bottom sponge portion 10b, with the protrusions 14 insertably engaging the corresponding wells 16. This causes at least some of the biological substance 22 to flow into the protrusions 14.

After the top and bottom sponge portions 10a, 10b are reconnected to form the biological substance-loaded sponge 10, the sponge 10 undergoes at least one soaking step. In the embodiment shown in FIG. 3, the entire sponge 10 is soaked 108 in saline solution, and then soaked 110 with additional biological substance solution to achieve a burst effect upon implantation into the patient, as soaking the sponge 10 in saline and biological substance solutions hydrates the collagen and starts the release of the growth factors inside the sponge. In alternate embodiments, the loaded sponge 10 may only undergo one of the soaking steps 108, 110, or no soaking step.

The soaked sponge 10 is then surgically implanted 112 into a pre-designated diseased or damaged area of the patient. After implantation, the biological substance 22 is released from the wells 16 through their pores 20, and through the pores 18 of the protrusions 14, and ultimately out of the sponge 10, into the patient's designated diseased or damaged area and the surrounding tissues, wherein it provides the positive therapeutic effect(s). In alternate embodiments, the sponge 10 has additional pores for the egress of the biological substance 22.

As discussed above, the number and size of the pores 18, 20 may vary in different embodiments, depending on the specific medical/therapeutic application. The number and size of the pores 18, 20 controls the release rate of the biological substance 22 and duration of its release.

It is to be expressly understood that the sponge 10 is suitable for use as part of, or in conjunction with, the systems and methods of the present disclosure, and is not limited to the examples described above, and that any type of sponge appropriate for a given application may be used in the systems and methods of the present disclosure.

While various embodiments of the present disclosure have been described in detail, it is apparent that modifications and alterations of those embodiments will occur to those skilled in the art. However, it is to be expressly understood that such modifications and alterations are within the scope and spirit of the present disclosure, as set forth in the following claims.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure. Moreover, though the present disclosure has included description of one or more embodiments and certain variations and modifications, other variations and modifications are within the scope of the disclosure, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. A delivery system for delivering at least one biological substance to a diseased or damaged area of a patient, comprising:
a sponge, including:
a top sponge portion 10a having a top base and a plurality of protrusions extending downwardly therefrom; and
a bottom sponge portion having a bottom base and a plurality of wells formed therein, the wells being configured to receive the at least one biological substance therein;
each of the plurality of wells being dimensioned to receive one of the plurality of protrusions therein upon placing the top sponge portion onto the bottom sponge portion to assemble the sponge.

2. The system of claim 1, wherein the at least one biological substance is contained in a solution.

3. The system of claim 1, wherein the at least one biological substance includes at least one growth factor.

4. The system of claim 3, wherein the at least one growth factor is selected from a group consisting of human recombinant growth factors, such as bone morphogenetic proteins (BMPs), fibroblast growth factors (FGF), insulin like growth factors (IGFs), platelet derived growth factors (PDGF), plasma rich growth factors (PRGF), transforming growth factor-β (TGF-β), epidermal growth factor (EGF), nerve growth factor (NGF) and vascular endothelial growth factors (VEGF).

5. The system of claim 1, wherein the at least one biological substance is selected from the group consisting of bone marrow aspirate and graft tissue.

6. (canceled)

7. The system of claim 1, wherein each of the protrusions has an outer surface having one or more pores therein, and each of the wells has a plurality of walls having one or more pores therein.

8. The system of claim 7, wherein the pores of the protrusions and wells have a pore size ranging from about 0.1 μm to about 1,500 μm.

9. The system of claim 7, wherein the pores of the protrusions and wells have a spacing between pores ranging from about 0.1 μm to about 2,000 μm.

10. The system of claim 1, wherein the sponge has a density ranging from about 0.05 g/cm³ to about 1.3 g/cm³,

11. The system of claim 1, further comprising at least one first collagen barrier layer overlying the walls of each of the plurality of wells.

12. The system of claim 11, wherein the at least one first collagen barrier layer is configured to control the release of the at least one biological substance from the plurality of wells.

13. The system of claim 11, further comprising at least one absorbent collagen layer overlying the at least one first collagen barrier layer.

14. The system of claim 13, wherein the at least one absorbent collagen layer is configured to control the release of the at least one biological substance from the plurality of wells.

15. The system of claim 11, wherein the at least one first collagen barrier layer has a hydrophilic/lipophilic balance (HLB) determined for controlling the diffusion of the at least one biological substance from the plurality of wells.

16. The system of claim 11, wherein the at least one first collagen barrier layer is hydrophilic.

17. The system of claim 11, wherein the at least one first collagen barrier layer is completely hydrophobic as to completely contain the at least one biological substance, and wherein the plurality of pores controls the diffusion of the at least one biological substance from the plurality of wells.

18. The system of claim 11, further comprising at least one second collagen barrier layer 28 overlying a surface of each of the plurality of protrusions.

19. The system of claim 18, wherein the at least one second collagen barrier layer is configured to control the release of the at least one biological substance from the plurality of protrusions.

20. The system of claim 11, wherein the at least one first collagen barrier layer 24 contains at least one glycosaminoglycan (GAG).

21. The system of claim 7, wherein the one or more pores in the plurality of walls of the wells are filled with a dissolvable material to delay the release of the at least one biological substance.

22. The system of claim 1, wherein the top sponge portion and bottom sponge portion are formed from collagen.

23. The system of claim 1, wherein the top sponge portion and bottom sponge portion are formed from a collagen-based composite.

24. The system of claim 23, wherein the collagen-based composite is selected from the group consisting of a collagen/mineral composite, a collagen/mineral/bioglass composite, a collagen/bioglass composite, a collagen/mineral/glycosaminoglycan composite, a collagen/mineral/bioglass/glycosaminoglycan composite and a collagen/bioglass/glycosaminoglycan composite.

25-43. (canceled)
